(19) United States
(12) Patent Application Publication
(10) Pub. No.: US 2015/0320352 A1
(43) Pub. Date: Nov. 12, 2015

(54) SYSTEM AND METHOD FOR PREVENTING DECUBITUS ULCERS

(71) Applicant: Enhanced Surface Dynamics, Inc., Palo Alto, CA (US)

(72) Inventors: Amir Ben Shalom, Modiin (IL); Lior Greenstein, Tel Aviv (IL); Arik Rofe, Ma'ale Hahamisha (IL)

(21) Appl. No.: 14/806,737

(22) Filed: Jul. 23, 2015

Related U.S. Application Data
(63) Continuation-in-part of application No. 13/264,036, filed on Jan. 4, 2012.
(60) Provisional application No. 61/202,848, filed on Apr. 13, 2009.
(60) Provisional application No. 61/296,967, filed on Jan. 21, 2010.
(60) Provisional application No. 61/304,507, filed on Feb. 15, 2010.

(51) Int. Cl. A61B 5/00; A61B 19/00

(52) U.S. Cl. CPC A61B 5/447; A61B 5/7445; A61B 5/6892; A61B 5/6891; A61B 5/6894; A61B 19/46; A61B 5/7275; A61B 2562/0247; A61B 2560/0252; A61B 2560/0257; A61B 2560/0475; A61B 2019/465

(57) ABSTRACT

A system to prevent the creation of pressure-wounds in a subject is provided, comprising at least one pressure detection mat comprising at least one layer of an insulating material sandwiched between first and second layers of conducting and a second layer of conducting strips overlapping at a plurality of intersections forming sensors, a driving unit configured to supply electrical potential selectively to the first layer' conducting strips, a control unit wired to the second layer's conducting strips and configured to control the driving unit and to receive data from the sensors, a processor configured to monitor electrical potential on the second layer's conductive strips to calculate impedance values and determine accumulated pressure applied thereto by calculating a summation of pressure measured by the sensor at predetermined intervals, and at least one display configured to present indications of pressure distribution and accumulated pressure.

### CROSS-REFERENCE TO RELATED APPLICATIONS

This application is a Continuation-in-Part of Applicant's co-pending U.S. patent application Ser. No. 13/264,036 filed Jan. 4, 2012, which is a national phase application under 35 USC 371 of International Patent Application No. PCT/IL2010/000294 filed Apr. 8, 2010, which claims the benefit of priority from U.S. Provisional Application No. 61/202,848 filed Apr. 13, 2009, U.S. Provisional Patent Application No. 61/296,967 filed Jan. 21, 2010 and U.S. Provisional Application No. 61/304,507 filed Feb. 15, 2010. The contents of all the above-referenced applications are incorporated herein by reference in their entirety.

### FIELD OF THE INVENTION

The present invention relates to pressure sensors. More particularly, embodiments described herein relate to medical aids for prevention of pressure-wounds such as decubitus ulcers or bedsores.

### BACKGROUND OF THE INVENTION

Pressure-wounds such as decubitus ulcers, which are commonly known as pressure ulcers or bedsores, are lesions developed when a localized area of soft tissue is compressed between a bony prominence and an external surface for a prolonged period of time. Pressure ulcers may appear in various parts of the body, and their development is affected by a combination of factors such as unrelieved pressure, friction, shearing forces, humidity and temperature.

Currently, about 10%-15% of hospitalized patients are estimated to have bedsores at any one time (Medicare website 2009). However, it is not only hospitalized patients who suffer from pressure-wounds: for example, people confined to wheelchairs are prone to suffer from pressure-wounds, especially in their pelvis, lower back and ankles Although easily prevented and completely treatable if found early, bedsores are painful, and treatment is both difficult and expensive. In many cases bedsores can prove fatal—even under the auspices of medical care.

The most effective way of dealing with pressure-wounds is to prevent them. Existing preventive solutions are either passive (e.g. various types of cushioning) or active, including a range of dynamic mattresses that alternate the inflation/deflation of air cells. Pressure relief mattresses however tend to re-distribute pressure also from locations where there was no need to relieve pressure thereby needlessly creating higher pressure in sensitive areas. Moreover, such mattresses are typically designed for patients lying down in hospital beds, and hardly answer the needs of individuals who spend considerable amounts of time sitting up, confined to a wheelchair or the like.

The most common preventive approach is keeping a strict care routine of relieving pressure off sensitive body areas of a patient every 2-3 hours. This can be done with patients under strict medical care. As well as being a difficult, labor intensive and costly task, such a care routine does not meet the needs of independent individuals who do not require ongoing supervision of a caretaker, such as paraplegics who use a wheelchair for mobility.

The need remains, therefore, for a reliable, cost effective system and method for preventing the development of pressure-wounds. Embodiments described hereinbelow address this need.

### SUMMARY OF THE EMBODIMENTS

Embodiments described herein disclose a pressure detection mat comprising a plurality of sensors configured to be placed between a subject and a platform and to couple with a pressure-wound prevention system.

Optionally, the pressure detection mat comprises at least one layer of an insulating material sandwiched between a first conductive layer and a second conductive layer. Optionally, the pressure detection mat further comprises at least one substrate layer. Optionally, at least one of the conductive layers are sandwiched between substrate layers.

Optionally, at least one of the conductive layers comprises parallel strips of conductive material. Optionally, the parallel strips of the first conductive layer and the parallel strips of the second conductive layers overlap at a plurality of intersections. Optionally, the parallel strips of the first conductive layer are arranged orthogonally to the parallel strips of the second conductive layer. Optionally, the intersections form capacitance sensors, resistance sensors or impedance sensors.

Optionally, the pressure detection mat further comprises attachment straps. Optionally, the pressure detection mat further comprises at least one humidity-detection sensor, or at least one temperature detection sensor.

Embodiments described herein further disclose a system configured to prevent the creation of pressure-wounds in a subject resting upon a platform, comprising at least one pressure detection mat, a driving unit configured to supply electrical potential to the pressure detection sensors comprising the pressure-detection mat, a control unit configured to control the driving unit and receive data from the sensors, a processor configured to interpret and analyze the data, and at least one display configured to present the data.

In the system, the pressure detection mat is optionally integral to a platform. Optionally, the platform is selected from a group consisting of mattresses, beds, chairs, stools, sofas, wheelchairs, rocking chairs, chaise longue, banquets, bean bags, ottomans, benches and poufs.

Optionally, the system further comprises at least one storage unit configured to store the data from the control unit and the processor. Optionally, the storage unit is mobile and configured to be integrated with a variety of pressure-detection devices. Optionally, the display is selected from a group comprising computer screens, laptops, Personal Digital Assistants, cellular phone screens, printed sheets, integrated Liquid Crystal Display screens, Thin Film Transistors (TFTs), touch screens and combinations thereof. Optionally, the processor uses configurable parameters to analyze the data.

Optionally, the system further comprises at least one sensor configured to monitor moisture. Optionally, the system further comprises at least one sensor configured to monitor temperature. Optionally, the system further comprises at least one sensor configured to detect contact between the subject and the platform. Optionally, the system is further configured to prevent a subject from falling off the platform.

Optionally, the system further comprises a unit configured to send data as to the system's whereabouts. Optionally, the system is further used to monitor the care routine of the subject. Optionally, the system comprises a plurality of pressure detection mats in communication with at least one common control center. Optionally, the system is used as a data harvesting research tool.

Embodiments further teach a method for preventing the development of pressure-wounds comprising providing at least one pressure detection mat comprising a plurality of sensors configured to detect pressure, supplying electrical potential to the sensors, receiving data from the sensors, interpreting and analyzing the data, and providing an output based upon the data. Optionally, the method further comprises storing the data in at least one data storage unit.

### BRIEF DESCRIPTION OF THE DRAWINGS

For a better understanding of the invention and to show how it may be carried into effect, reference will now be made, purely by way of example, to the accompanying drawings.

With specific reference now to the drawing in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention; the description taken with the drawing making apparent to those skilled in the art how the several forms of the invention may be embodied in practice.

In the accompanying drawings,

FIG. 1a shows the main components of a general embodiment of a pressure-wound prevention system;

FIG. 1b shows an extended pressure-wound prevention system including a plurality of pressure-wound prevention sub-systems of different kinds;

FIG. 2a shows a cross section of an embodiment of a single sensor; and

FIGS. 2b-e show various isometric projections of embodiments of a pressure-detection sheet;

FIGS. 3a-b show a top view and a section through view of a further embodiment of a pressure detection sheet;

FIG. 4 shows a pressure-wound prevention system incorporated into a wheelchair;

FIGS. 5a-d show various representations of how pressure data may be displayed on a screen of an embodiment of display system;

FIG. 6 is a flowchart of a method for preventing the development of pressure wounds;

FIG. 7 is a flowchart showing a possible method for presenting pressure data related to the risk of a subject developing pressure injuries

FIG. 8 is a flowchart of another method for determining the risk of a subject developing a pressure injury;

FIGS. 9a-f show a selection of some common postures, which, amongst others, may adopted by subjects recumbent upon a horizontal surface;

FIGS. 10a and 10b are graphical illustrations of a coordinate system of the pressure sensing assembly and a coordinate system of a subject body respectively; and

FIGS. 11a and 11b represent a possible pressure distribution image and an associated body model, respectively, showing pressure distribution for a subject in a supine posture.

### DETAILED DESCRIPTION OF THE SELECTED EMBODIMENTS

Embodiments of the pressure detection system and method described hereinbelow, are directed towards preventing pressure-wounds from developing in a subject. The embodiments generally provide a caretaker with indications of pressure distribution and ongoing, accumulated pressure exerted upon body parts of a subject, which may result in the creation or progression of a bedsore. A caretaker may then take appropriate action, such as to move the subject or change his cushioning in a way that relieves pressure upon the effected body part. Embodiments of the system may also be used for ongoing analysis and recording of a subject's care routine.

It will be appreciated that embodiments of the pressure-detection system allow a caretaker to move the patient only when it is needed. Furthermore, attention may be targeted towards the pressured part of the body specifically, which may be repositioned or cushioned as required. It is further noted that embodiments of such a system may further assist in monitoring a subject's care routine and his caretaker's performance.

Various embodiments of the system and method for preventing pressure-wounds are presented hereinbelow. Typically, they utilize pressure-detection elements to determine which areas of a subject's body are at risk of developing pressure ulcers. One of these elements could be a pressure-detection sensing mat, configured to couple with a pressure-wound prevention system as outlined below.

### Pressure-Wound Prevention System Including a Sensing Mat

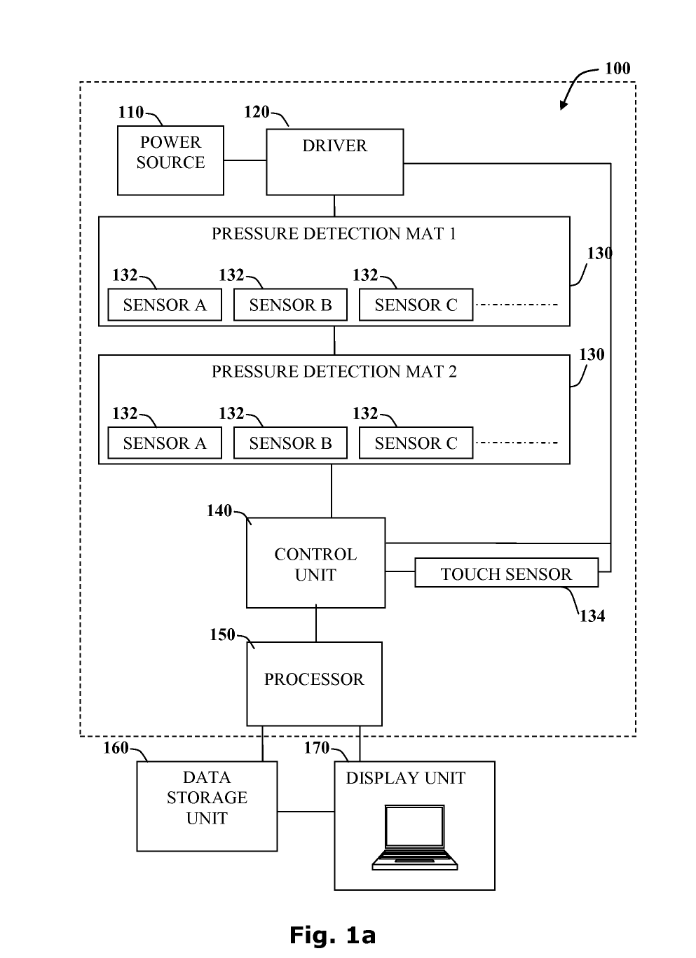

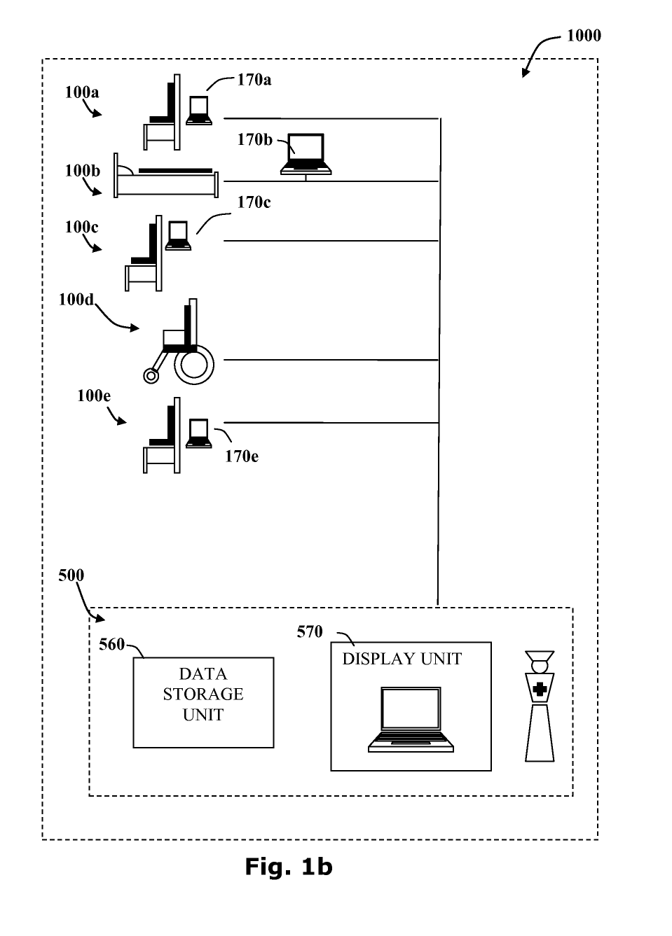

Reference is now made to the block diagram of FIG. 1a, representing the main components of a general embodiment of a pressure-wound prevention system 100. Embodiments of such a system may include at least one pressure-detection mat 130 comprising a plurality of sensors 132, a driver 120, a control unit 140 typically connected to a power source 110, a processor 150, a data storage unit 160 and a display system 170. The system may optionally include additional sensors such as a touch sensor 134 configured to detect contact between a platform and a subject's body. In this embodiment, the driver 120 selectively supplies voltage to sensors in the pressure-detection mat and optionally to the touch sensor 134. The processor 150 monitors the potential across the sensors in the pressure detection mat, calculates impedance values for each sensor, and stores that data in a data storage unit 160. The processor optionally monitors data received from the touch sensor as well. The stored data may be further processed, analyzed, and displayed on a display system 170, such as computer screens, laptops, PDAs, cellular phone screens, printed sheets, integrated LCD screens (e.g. Thin Film Transistors, touch screens) and the like. Although presented in the block diagram as separate blocks, the system may optionally be integrated into a stand-alone system.

Measurement readings from the multiple sensors of the pressure-detection mat may be transmitted to a processor 150. Data transmission may be wireless or via data cables according to requirements. The processor 150 may be configured to interpret impedance values and to analyze the data to determine which sensors had pressure applied to them, and to correlate the impedance with a pressure value, thereby facilitating the system 100 to measure the pressure applied to each of the sensors (for convenience, this process may be referred to herein as a pressure measurement by the sensor). The pressure values determined may be stored in the data storage unit 160, as described herein. The interpretation may be performed by consulting with a lookup table which maps impedance values at a given frequency to pressure values, typically in units of millimeters of mercury, as commonly used in medical settings, although other pressure units such as pascals, atmospheres, pounds per square inch or the like may be preferred as suit requirements. The values in such a lookup table will typically differ from one mat to another, and may need to be calibrated automatically or manually, possibly during manufacture or upon initial usage of the mat. It will be appreciated that impedance measurements are effected by a number of properties of the sensors such as resistance, capacitance and inductance, any of which may indicate pressure according to the configuration of the sensing mat.

The processor 150 may be further configured to associate stored data, e.g., in the data storage unit 160, with an individual subject, irrespective of the pressure-detection mat 130 used to collect it. In addition, it may be configured to map each sensor to an area of the subject. Accordingly, e.g., different pressure-detection mats 130 may be used with a single subject (e.g., the subject may be moved among different pressure-detection mats, such as on different beds in a care facility), with the processor 150 relating data collected by the different pressure-detection mats to single subject, and the mapping information facilitating associating pressure measured by sensors on one pressure-detection mat 130 with corresponding (i.e., measuring pressure at the same area of the subject) sensors on another pressure-detection mat.

In addition to facilitating monitoring a single subject from multiple pressure-detection mats 130, the processor 150 may utilize information about a single subject from multiple pressure-detection mats to assist a caretaker in tracking a subject, in particular one who is relatively independent. For example, several pressure-detection mats 130 may be located in different areas (e.g., in a subject's home, on his bed, sofa, and easy chair). The processor 150 may thus be configured to provide information to a caretaker, e.g., in realtime, in the form of periodical reports, etc., regarding a subject's location based on when information is collected be each of the pressure-detection mats 130. According to some examples, the processor may be configured to provide information (e.g., all information, alerts, etc.) to a caretaker via a remote device, such as a mobile phone, web interface, etc.

The processor 150 may be further configured to identify a subject based on a pressure profile detected by the sensors. For example, it may develop and maintain a “subject library,” comprising pressure profiles of different subjects. Accordingly, the processor 150 is configured to analyze the pressures measured by the sensors when a subject sits or lies on a pressure-detection mat 130, determine if it is consistent with any of the pressure profiles in the subject library, and thereby automatically identify the subject on the pressure-detection mat. The processor may be further configured to take one or more predetermined actions based on the identified subject. The actions may include, but are not limited to, one or more of adjusting ambient conditions (lights, temperature, etc.), notifying a caregiver of the subject's presence, alerting the subject (e.g., based on the identified subject and optionally other factors, such as time of day, a reminder that the subject should do a predetermined activity), etc.

According to some modifications, the pressure-detection mat 130 may be configured for use on the floor, i.e., to measure the pressure exerted by a subject walking thereon. According to these modifications, the processor 150 may be configured to determine a “gait profile” of a subject walking across it, and identify a subject by comparing it to a library of stored gait profiles. The gait profile may comprise, but is not limited to, one or more of the subject's speed, pressure profile of each foot, change in each pressure profile over the course of a step, and stride length.

The processor 150 may be further configured to determine an accumulated pressure applied over time to each of the sensors. The accumulated pressure may be a summation of pressure measured by a sensor at predetermined intervals. For example, the processor may determine the accumulated pressure at a given sensor by adding the pressure measured thereby every 5 seconds. The processor may continue the summation indefinitely, or over a predetermined time period (such as adding the pressure measured by a sensor every 5 seconds over a period of three minutes; thus, the processor may be configured to generate a history of accumulated pressures for a given sensor).

The processor 150 may be further configured to take selective action based on pressure and/or accumulated pressure detected. For example, the processor 150 may be functionally connected to massage and/or heating elements to direct their operation. The massage/heating elements may constitute part of the pressure-detection mat 130, or be separate therefrom, for example comprising portions of a separate mat. The processor 150 may be configured to determine, based on pressure and/or accumulate pressure, that one or more areas on the subject's body could benefit from massaging and/or heating, for example to reduce the risk of pressure wounds such as decubitus ulcers, and direct operation of the appropriate elements accordingly.

The processor 150 may be further configured to use measured/calculated parameters to assess the risk of a subject developing pressure wounds, such as decubitus ulcers, or one or more other conditions owing to pressure which may develop (and remain unmitigated) under a subject. The parameters may include, but are not limited to, one or more of instantaneous pressure, accumulated pressure, sudden changes in pressure, etc. Accordingly, the processor may be preloaded with information correlating relevant parameters with such conditions.

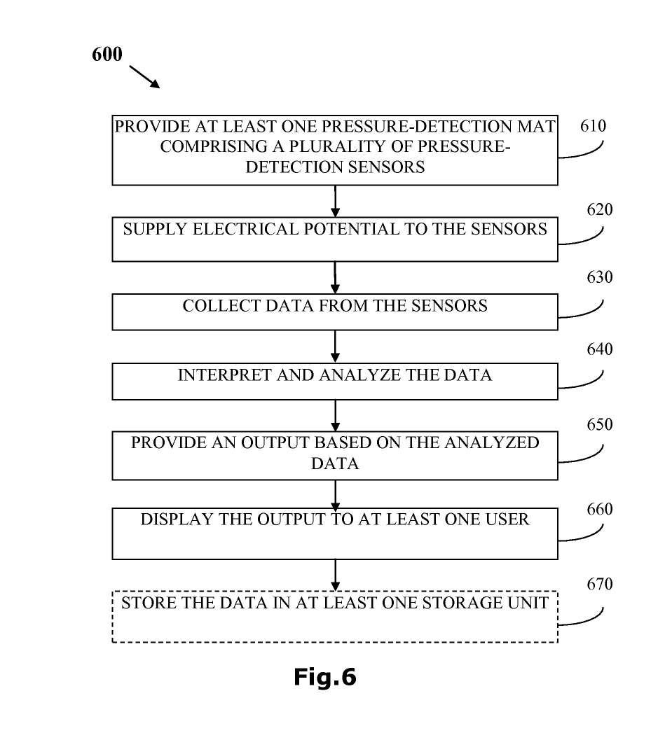

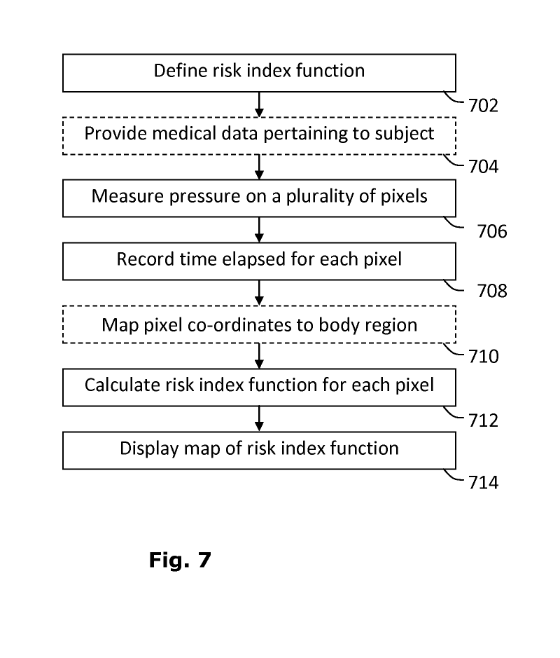

As illustrated in FIG. 7, a method is provided for determining and displaying pressure related measurements. The method may be carried out by the processor 150 using pressure values measured by the sensors to generate values of risk index and to indicate these on a map.

The method may include defining a risk index function—step 702. The risk index function may be a function such as the accumulated pressure risk factor R₂=PΔt, based on the product of pressure exerted P with the time Δt during which the pressure was recorded, as described hereinabove. Alternatively, the risk index function may consider other relevant factors such as tissue type, condition of patient, region of the body and the like. Accordingly, relevant medical data pertaining to the subject may be provided to the system—step 704.

The pressure may be measured by a plurality of pressure sensing elements—step 706. Such data may be recording using the pressure-detection mat 130, or any other suitable apparatus. The time elapsed during which pressure is measured for each pressure sensing element may be recorded—step 708.

Optionally, the pixel coordinates may be mapped onto a two dimensional array, to the plurality of pressure sensing elements. Alternatively, the pixel coordinates may be mapped to a body-based coordinate system—step 710. The body based coordinate system may allow the risk index to be calculated for each region of the body, which may be relevance to some defined risk index functions as described hereinabove.

A value for the risk index function may be calculated for each pixel—step 712. It is noted that values may be calculated for each pressure sensing element, based on the pressure measured and the time elapsed, in a two dimensional matrix and/or for points on the body coordinate system. The risk indices may be presented as a map—step 714. The map displayed may provide an ongoing record of ongoing risk of a subject developing pressure related injuries which is in a form readily accessible to a caregiver.

Since an injury prevention system may be configured to detect pressure ulcers over the surface of a patient's body, the processor 150 may be configured to calculate a pixel coordinate based risk index function, associating risk with points upon the supporting surface, such as a mattress or the like:

$$r ( τ , x , y ) = {\sum }_{t = 1}^{τ} K ( t - τ ) * p ( P ( τ , x , y ) ) * {s}_{u} ( x , y , w , a )$$

wherein:
K (t−τ) is a time kernel function representing the additive effects of pressure;
p(P(τ, x, y)) is a pressure risk index, which may be considered as a function of pressure P measured at a given point (x, y) and at a given time τ; and
sᵤ(x, y, w, a) represents the sensitivity of body tissue of the subject at a given point (x, y), and which may depend upon a range of medical influences, such as the subject's weight w and age a.

Alternatively, the processor 150 may be configured to calculate the risk index function in a body coordinate system, rather than a mattress coordinate system, as follows:

$$r ( τ , {x}_{u} , {y}_{u} ) = {\sum }_{t = 1}^{τ} K ( t - τ ) * p ( P ( τ , {x}_{u} , {y}_{u} ) ) * {s}_{u} ( {x}_{u} , {y}_{u} , w , a )$$

where each point on the body surface may be represented by a body coordinate vector (xᵤ,yᵤ).

Accordingly, a risk transform may be defined to measure the possibility of pressure injuries such as stress sores developing. Risk index values may measure the risk that a particular region of interest may develop a pressure injury. The size of each region of interest may be defined by the limit of the resolution of the data collected. Where data is collected by a pressure sensing assembly such as described herein, the smallest region of interest may be defined by the size of the pressure sensing elements, for example the intersections of the conducing strips in a pressure sensing mattress.

The risk transform may be used to generate solutions for the problem and may further develop more accurate formulas based on probabilistic theory. Accordingly, methods for describing the state of risk for a subject, such as visual methods for displaying the data or analytical methods for calculating a state of risk for a subject, may present monitored pressure data as a risk transform. This may enable a standardization of the analysis and the presented data. Moreover value standardization may enable the ready comparison between different methods.

The risk transform may be unit-less, having values referring to probabilities or pseudo-probabilities. For an initial calculation, the risk may be formulated as an approximate probability without necessarily preserving a full probabilistic formulation of the risk. Methods are presented which transform pressure values as measured, for example in millimeters of mercury, pascals, pounds, newtons or the like, to the risk transform in order to determine the risk of a region of interest developing a pressure injury such as a stress sore.

In one model, it may be assumed that occurrence of a pressure injury is a stochastic variable with a probability ‘r’ as defined by the risk index function. A probabilistic model may be simplified by assuming that all of the variables in the model, with the single exception of ‘occurrence of a pressure injury event’ (PSE) may be independent stochastic variables. Accordingly, using a Bayesian model, the risk index function r may be represented by:

$$r ( τ , {x}_{u} , {y}_{u} ) = {Pr}_{τ , {x}_{u} , {y}_{u}} ( PSE \vert P ( τ , {x}_{u} , {y}_{u} ) , u , w , a , r ( τ - dt , {x}_{u} , {y}_{u} ) ) = \frac{\begin{matrix}{Pr}_{τ , {x}_{u} , {y}_{u}} ( P ( τ , {x}_{u} , {y}_{u} ) \vert PSE ) {Pr}_{τ , {x}_{u} , {y}_{u}} ( u \vert PSE ) \\ {Pr}_{τ , {x}_{u} , {y}_{u}} ( w \vert PSE ) {Pr}_{τ , {x}_{u} , {y}_{u}} ( a ) {Pr}_{τ , {x}_{u} {y}_{u}} ( r ( τ - dt , {x}_{u} , {y}_{u} ) )\end{matrix}}{\begin{matrix}{Pr}_{τ , {x}_{u} , {y}_{u}} ( P ( τ , {x}_{u} , {y}_{u} ) ) {Pr}_{τ , {x}_{u} , {y}_{u}} ( u ) {Pr}_{τ , {x}_{u} , {y}_{u}} ( w ) \\ {Pr}_{τ , {x}_{u} , {y}_{u}} ( a ) {Pr}_{τ , {x}_{u} , {y}_{u}} ( r ( τ - dt , {x}_{u} , {y}_{u} ) )\end{matrix}}$$

Such a formula may be used to generate probabilistic estimations of the risk of each region of interest developing a pressure injury given current pressure conditions. Empirical data regarding the probability of pressure injury occurrence for various conditions may be gathered in preliminary data collection operations or accumulated over time. Such empirical data may be embedded in the formula in order to obtain the required risk index for each point on the surface of a body.

According to one algorithm, risk may be measured by recording a pressure distribution image of a subject, identifying the posture of the subject, mapping the pixels of the pressure sensing apparatus to a body coordinate system and calculating the risk of developing pressure injuries for each point on the body coordinates according to a formula such as the one outlined above.

The accumulated risk index may be presented to a caregiver as a visual display, for example on a body model, a rectangular array, a pressure distribution map or the like. It is particularly noted that a common risk index parameter may summarize the pressure risk values on the surface of a subject possibly facilitating the quantification and analysis of a subject's condition by a caregiver.

Various simplifications may be used to enable the assignation of risk index for monitored pressures. For example a non-linear relationship may be defined between pressure and risk or a pressure threshold may be established above which the accumulated pressure may be deemed high risk. Additionally or alternatively, a sigmoid weighting threshold function, W_{g}, may be used to adjust the pressure or any risk estimation by a multiplication between the values.

Accordingly, a single parameter measurement, the total risk R, may be calculated using the formula

$$R ( t = T ) = {\sum }_{x , y} {risk}_{t} ( x , y ) * {Wg}_{t} ( x , y )$$

The above described functions may be determined by experimental data. Such experimental data may be harvested, at least in part, from existing published literature. The frequency of the development of stress ulcers and the like at various areas of the body may be counted for subjects of various ages, weights, and genders as well as for different medical conditions, all of which may be included in more detailed risk factor functions. Thus a probabilistic model may be generated. Furthermore, additional data may be gathered from ongoing monitoring of subjects using pressure sensing systems. It will be appreciated that, over time, as the data reservoir grows, the accuracy of the risk factor functions may be improved.

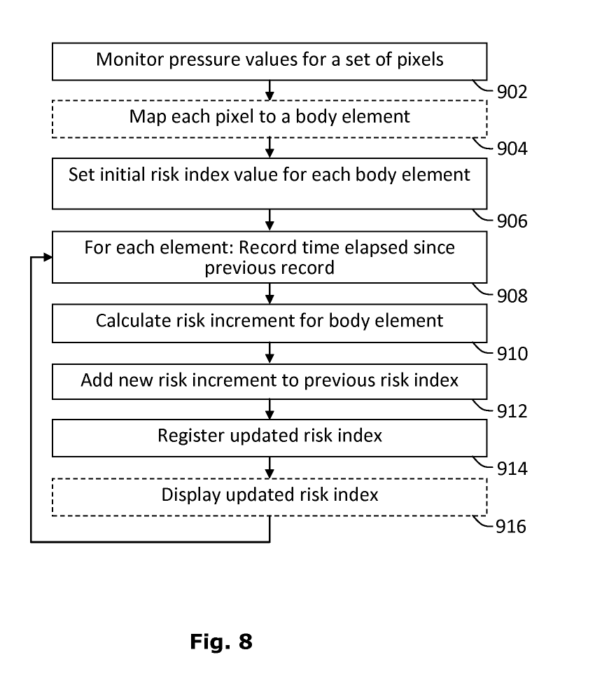

Referring now to the flowchart of FIG. 8, a method is presented for determining the risk of a subject developing a pressure injury. The method includes monitoring pressure values for a set of pixels—step 902, for example, pressure may be measured using a set of pressure sensing elements corresponding with an area of overlap between a subject and a pressure sensitive sheet. Optionally, each of the pixels for which a pressure value has been monitored may be mapped to a body element—step 904.

An initial risk index may be set for each pixel or body element—step 906. After a certain duration, the time elapsed may be recorded—step 908 and a risk increment may be calculated for the pixel—step 910. The risk increment may be a function of the time elapsed and the pressure recorded during the elapsed time.

The risk increment may be added to the previous risk index to provide a new risk index—step 912. This risk index may be registered—step 914, for example by saving its value to a database of risk index values or the like.

Where appropriate, the risk index may be presented upon a visual display—step 916, perhaps using a color coded pressure risk map, a projection of pressure risk value representations onto a body model or the like. Such a display may provide a caregiver with an intuitive indication of risk of a subject developing pressure injuries and of possible preventative actions which may be taken to avoid such injuries developing.

As noted above, for various applications, it may be useful to identify body posture of the patient. Such identification may enable body features to be recognized or a body coordinate system to be mapped. It is noted that by recording a series of body postures adopted by a subject, it may be possible to determine other factors such as the risk of the subject falling from a bed or the like. Furthermore, knowing a subject's posture history may assist caring staff such as nurses or the like to choose a suitable new posture in which to reposition the subject when necessary.

Recumbent postures may be broadly classified by the orientation of the subject such that a posture where a subject is lying on her back may be termed a supine posture, a posture where a subject is lying on her front may be termed a prostrate posture, a posture where a subject is lying on her left side may be termed a left leaning posture and a posture where a subject is lying on her right side may be termed a right leaning posture.

Referring now to FIGS. 9a-f, six body profiles are shown representing a selection of common postures adopted by subjects recumbent upon a horizontal surface. The postures shown illustrated some general posture classes adopted during sleep. FIG. 9a shows a right leaning posture known as ‘foetus’ which is adopted by about 41% of recorded sleepers. It will be appreciated that an equivalent left leaning ‘foetus’ posture may also be adopted. FIG. 9b shows a left leaning posture known as ‘log’ which is adopted by about 15% of recorded sleepers. It will be appreciated that an equivalent right leaning log posture may also be adopted. FIG. 9c shows a left leaning posture known as ‘yearner’, which is adopted by about 13% of recorded sleepers. It will be appreciated that an equivalent right leaning yearner posture may also be adopted. FIG. 9d shows a supine posture known as ‘soldier’, which is adopted by about 8% of recorded sleepers. FIG. 9e shows a prostrate posture known as ‘freefaller’ which is adopted by about 7% of recorded sleepers. FIG. 9f shows a supine posture known as ‘Starfish’ which is adopted by about 5% of recorded sleepers. It will be appreciated that further postures may be adopted, for example in hospital environments where subjects may have various injuries or ailments making adoption of common postures difficult or impossible.

It is noted that methods and systems of the disclosure may be able to identify such general posture classes. Furthermore each of the general posture classes listed above may include multiple variations. For example, a subject may lean to the right or the left, limbs may be shifted to various angles, and the head may be turned to right or left and the like. Systems and methods described herein may be utilized to distinguish between these variant postures and posture categories. By identifying postures, the position of the limbs may be identified and pixels may be mapped to a body coordinate system as required.

As noted above, for various applications, it may be useful to identify body posture of the patient. Such identification may enable body features to be recognized or a body coordinate system to be mapped. It is noted that by recording a series of body postures adopted by a subject, it may be possible to determine other factors such as the risk of the subject falling from a bed or the like. Furthermore, knowing a subjects posture history may assist caring staff such as nurses or the like to choose a suitable new posture in which to reposition the subject when necessary.

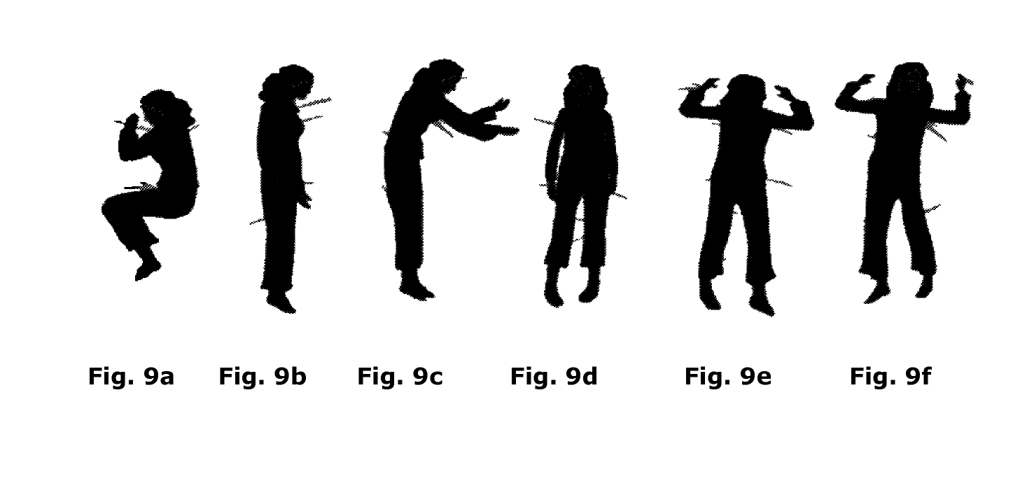

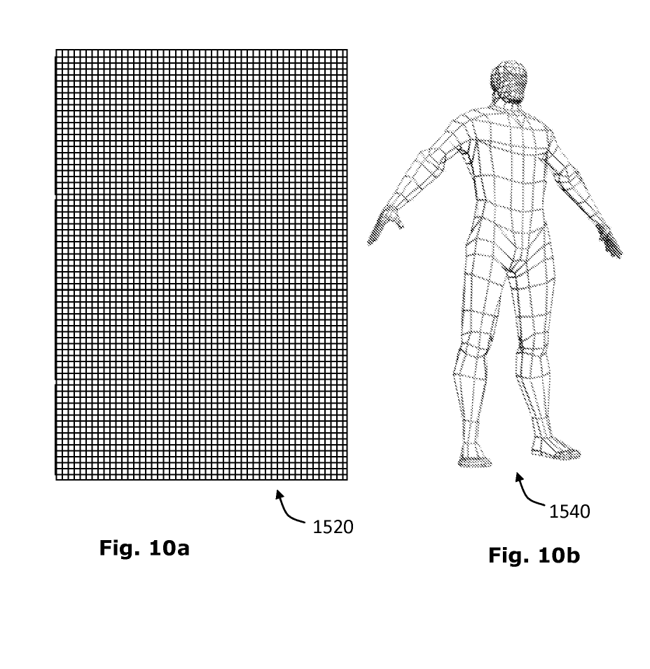

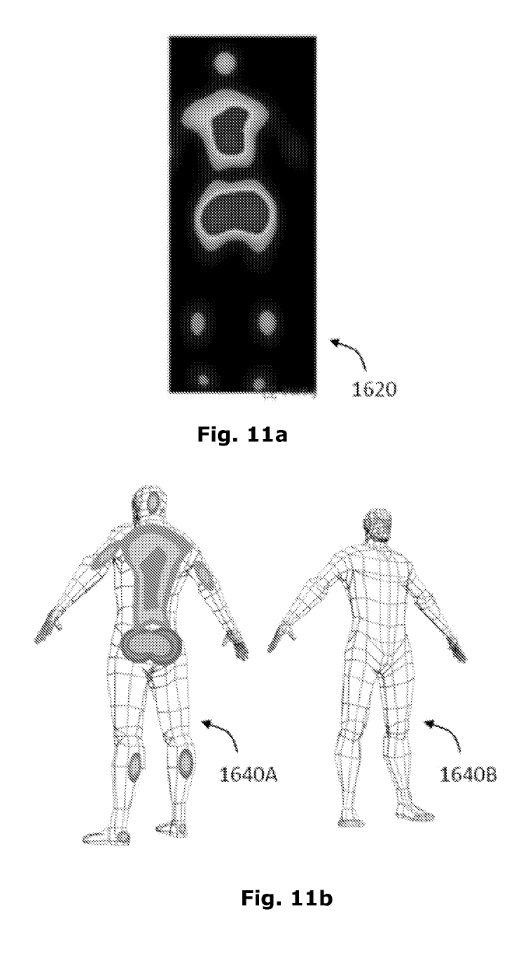

Referring to FIGS. 10a and 10b graphical illustrations are presented representing, respectively, a coordinate system of the pressure sensing assembly 1520 and a coordinate system of a subject body 1540. The pressure sensing elements of the pressure sensing assembly may be arranged as a two dimensional surface 1520, possibly as a rectangular arrangement or the like. The mapping of this two dimensional surface 1520 to a subject body coordinate system 1540 is a complex procedure.

The subject body is a three dimensional structure and the surface in contact with the pressure sensing elements is of two dimensions. However, the contact area between the subject body and the pressure sensing assembly may change with the movements of the subject, or movements of the pressure sensing assembly itself. Thus different pressure images and different associated postures may require different mappings between points on the body surface and sensing elements.

A pressure distribution image as recorded by the pressure sensing assembly may represent the pressure P exerted by the subject as measured by each of the pressure sensing elements. Each pressure sensing element may be situated at a known point which may be represented by a location vector in the coordinate system 1520 of the pressure sensing assembly. Accordingly, the recorded pressure value for the element may be associated with the location vector in the coordinate system 1520 of the mat. The location vector associated with each pressure value of the pressure distribution image coordinate system may be transformed to a mapped vector in a subject based coordinate system 1540.

The transformation from a location vector in the coordinate system of the mat 1520 to a mapped vector in the subject based coordinate system 1540 may require the identification of the current posture. This may allow landmark body points, to which a body coordinate system may be anchored, to be determined. Current posture may be identified, for example, using known algorithms such as particle component analysis, support vector machine, K-mean, two-dimensional fast Fourier analysis, earth movers distance and the like. In particular the earth mover distance (EMD) algorithm is a method to compare between two distributions, and which is commonly used in pattern recognition of visual signatures. The EMD algorithm may be readily applied, for example, to compare between a recorded posture and candidate posture types stored in a database. It is noted that the identification of particular body regions may have further application, for example in enabling a pressure wound prevention system to associate a particular pressure value with the relevant body region for more accurate calculation of a risk index function for that point.

In order to display any time dependent measurement which relies on body-local pressure values, it may be useful to track a body region of interest over time. Accordingly, it may be useful to detect such body regions of interest so that recorded pressure values and their changes over time may be associated therewith.

In general an algorithm may be used to receive an input of an array, possibly a rectangular array 1520, of pressure values from a pressure sensing assembly; and to return an output of pressure values associated with body coordinates 1540.

FIGS. 16a and 16b represent a possible pressure distribution image 1620 as recorded by a pressure sensing assembly and an associated body model 1640A, 1640B (referred to hereinafter collectively as 1640), respectively. The pressure distribution image 1620 may be collected, for example, when a subject is recumbent upon a surface in a supine posture, possibly identified as ‘soldier’ for example. The body model may have a back aspect 1640A and a front aspect 1640B.

A body model 1640 may be defined, for example, by identifying the posture of the subject from the pressure distribution image 1620 and identifying key body features from the identified posture. The body model 1640 may be used to calibrate the system by associating each pixel of the pressure distribution image 1620 corresponding to a point of contact between the pressure sensing assembly and the subject with a region on the surface of the subject's body.

Once the pixels are mapped to the body surface, the corresponding pressure records or calculations may be associated with the relevant body regions and the pressure distribution image 1620 may be reconstructed by projecting the pressure values of each pixel onto the body model 1640. It will be appreciated that as the subject position changes or a new posture is identified, the mapping may be recalibrated. Accordingly, where appropriate, pressure records may remain associated with the same body regions over time even when data is collected from different pressure sensing elements.

### Extended System for Multiple Subjects

Other embodiments of the pressure-wound prevention system can be designed for scale and stress, aiming to monitor the accumulated pressure on a plurality of subjects. Such embodiments may include a plurality of pressure-detection mats connected to one or more drivers and control units. Power may be supplied from a plurality of sources, multiple processors may be used for calculation and analysis of the data, which may be stored in a plurality of data storage units.

Reference is now made to FIG. 1b, showing an extended pressure-wound prevention system 1000 including a plurality of pressure-wound prevention sub-systems 100a-e in communication with a common remote control center 500. The pressure-wound prevention sub-systems 100a-e may monitor various subjects in various positions for example on beds 100b, chairs 100a, 100c, 100e and wheelchairs 100d in a hospital, care home or the like and may be configured to communicate with a remote control center 500 for example at a nursing station via a data communication line. It will be appreciated that in embodiments where the pressure detection mat is configured to move such as where the subject is seated in a wheelchair or the like, wired data cables may be inappropriate and data transmission via wireless means may be preferred, for example via radio waves using protocols such as wifi, Bluetooth or the like.

Alternatively, the plurality of pressure-wound prevention sub-systems 100a-e may be located remotely from one another for example each in an individual home, and the remote control center 500 may be a manned monitoring station for the purpose. In such systems, a data communication line may be provided via a cellular network, connections to the internet or the like.

It is further noted that a single pressure-wound prevention system may include multiple pressure detection mats, for example and without limitation two mats located on a seat of a chair and on a back of a chair.

The remote control center 500 typically includes a data storage unit 560 for storing data from the sub-systems 100a-e and a display unit 570 for presenting the data as required. It will be appreciated that the control center 500 may additionally provide processing and driving functionality for controlling multiple sub-systems. Optionally each pressure-wound prevention sub-system 100a-e may have its own dedicated monitor 170 for processing, storing and displaying data locally.

Pressure Sensing Mat for Use with Pressure-Wound Prevention Systems

Embodiments of a pressure sensing mat are disclosed. The sensing mat may be placed between a seat of a chair or a mattress of a hospital bed and the body of a seated subject. The sensing mat is typically used to monitor the pressure exerted upon the subject in a sitting or lying position. The output of the pressure sensing mat may be used to indicate the risk of pressure-wound development.

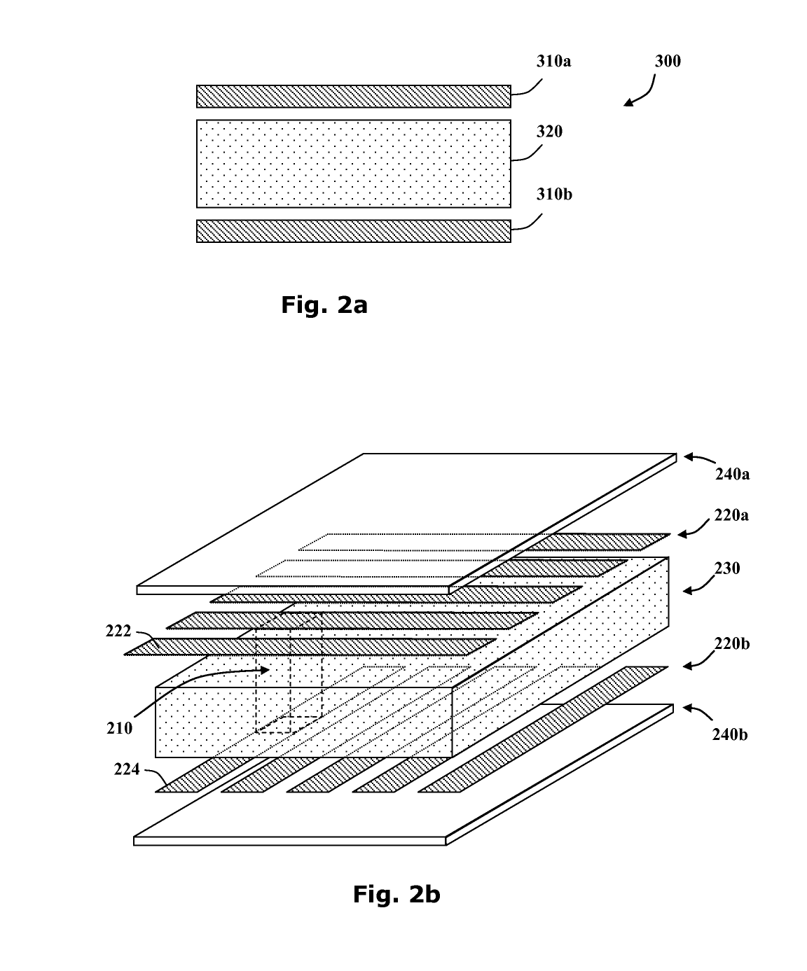

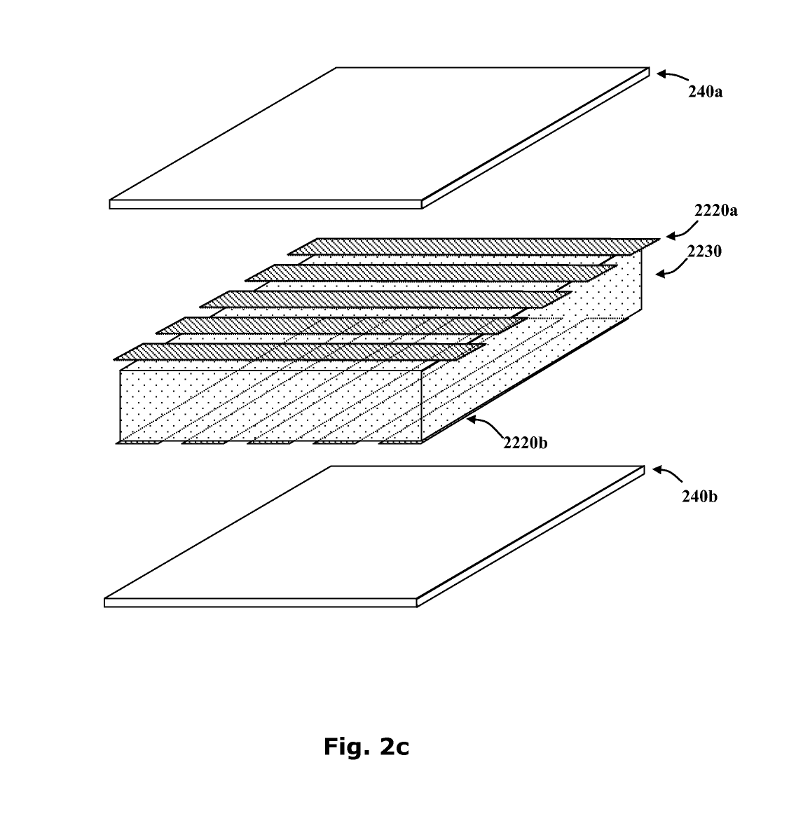

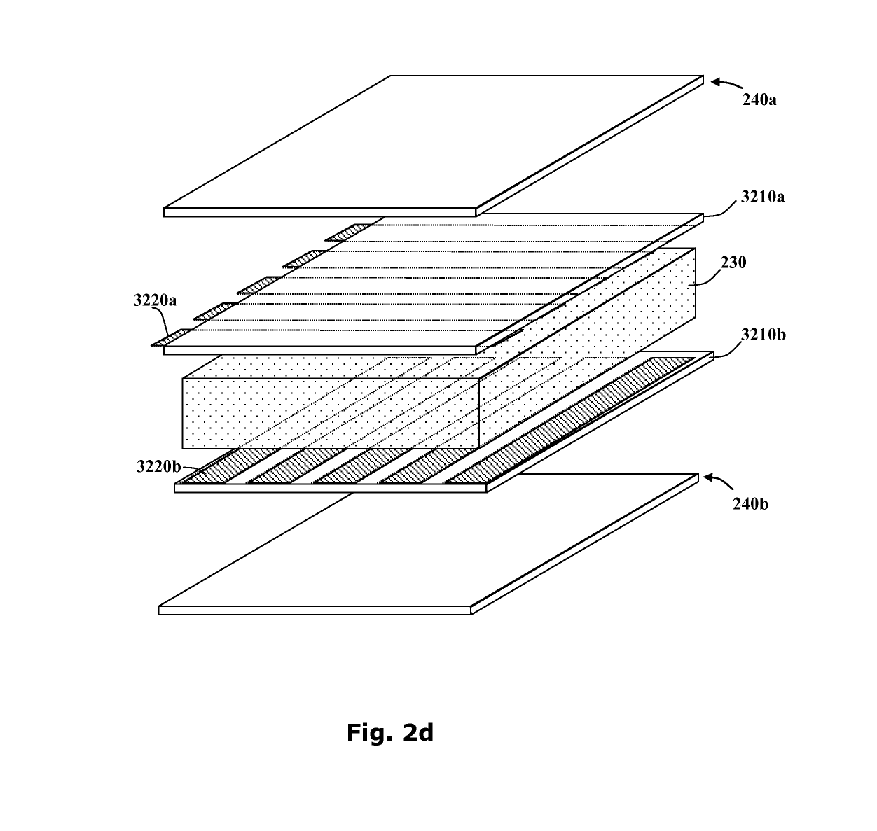

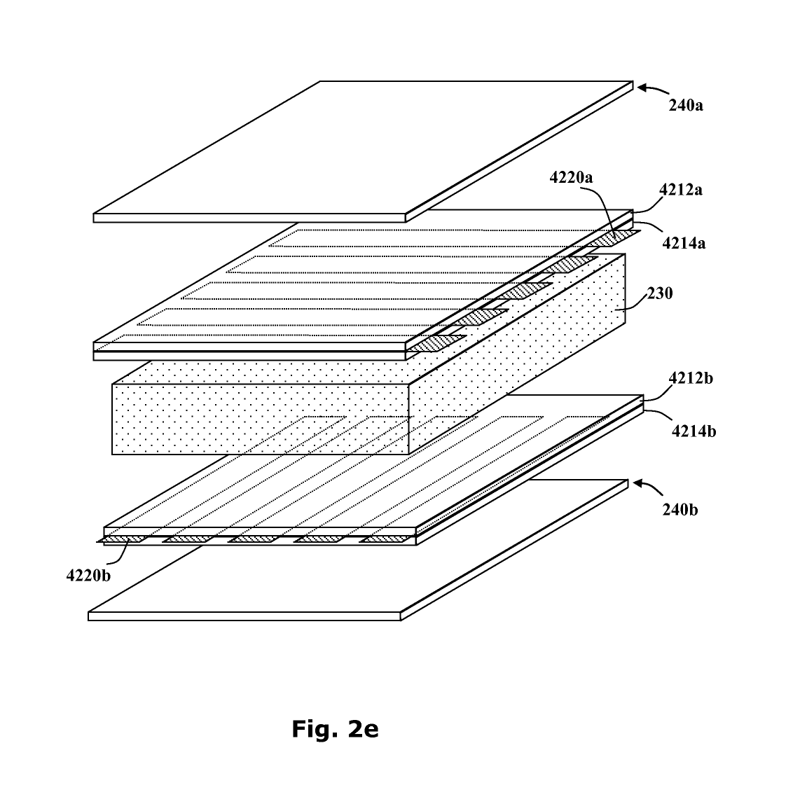

Reference is now made to FIG. 2a, showing a cross section of a basic embodiment of a single sensor 300. In this embodiment, the sensor is a capacitor comprised of two layers of conductive strips 310a, 310b and an insulating layer 320 of isolating material therebetween. Pressing anywhere on the sensor would compress the insulating layer 320 changing the distance between the conductive strips and thereby changing the capacitance of the capacitor. Although only a capacitance sensor is described, it is noted that according to other embodiments, resistance sensors may be preferred. Accordingly, the resistance of the insulating layer may be monitored as it varies according to pressure.

Reference is now made to FIG. 2b showing an isometric projection of an embodiment of a pressure-detection mat 200 comprising a plurality of sensors 210 arranged in a form of a matrix. The mat typically has two layers 220a, 220b of conductive material separated by an insulating layer 230 of isolating material. Each of the conductive layers typically consists of parallel conductive strips 222, 224 and the two conductive layers are arranged orthogonally such that in one conductive layer the strips are horizontal 222 and in the other conductive layer they are vertical 224. Each strip is wired to a control unit and is preferably operable by safe low voltage source.

A capacitance sensor is based on the capacitance between the sections of the conducting strips overlapping at each “intersection” of a vertical conductive strip with a horizontal conductive strip. These capacitance sensors are configured such that pressing anywhere on their surface changes the spacing between the two conductive layers, and consequently the capacitance of the intersection. A driving unit may selectively provide an electric potential to the vertical strip and the electrical potential may be monitored on the horizontal strip such that the capacitance sensor of the overlapping section may be determined.

It is noted that by providing an oscillating electric potential across each sensor and monitoring the alternating current produced thereby, the impedance of the intersection may be calculated and the capacitance of the intersection determined. The alternating current varies with the potential across a capacitor according to the formula:

I_{ac}=2πfCV_{ac}

where I_{ac} is the root mean squared value of the alternating current, V_{ac} is the root mean squared value of the oscillating potential across the capacitor, f is the frequency of the oscillating potential and C is the capacitance of the capacitor.

Thus where the values of V_{ac} and I_{ac} are known at a known frequency, the capacitance of a sensor may be calculated. Accordingly, where the mechanical properties of the sensor are known, the pressure applied upon the sensor may be deduced.

Preferably a capacitance sensor will retain its functionality even if it is fully pressed continuously for long periods such as or even longer than 30 days and keep its characteristics for periods over the lifetime of the sensing mat which is typically more than a year. Notably, the sensor characteristics should preferably be consistent between two separate events.

According to some embodiments, the mat may further include additional sensors configured to monitor additional factors, particularly additional factors influencing the development of bedsores, such as temperature, humidity, moisture, or the like. Such additional sensors may be configured to monitor the factors continuously or intermittently as appropriate to detect high risk combinations of factors. Such measurements may be recorded and stored in a database for further analysis.

Optionally, additional sensors may be located apart from the pressure-detection mat. For example, the mat could be integrated into a seat of a chair and a touch sensor could be integrated into a chair's back support.

In preferred embodiments of the pressure-detection mat, the materials are selected such that the conductive layers and insulating layers are flexible. The insulation material may be a compressible typically sponge-like, airy or poriferous material (e.g. foam), allowing for a significant change in density when pressure is applied to it. Materials comprising the sensing mat are typically durable enough to be resistant to normal wear-and-tear of daily use. Furthermore, the sensing mat may be configured so as not to create false pressure readings for example when the mat is folded.

The pressure-detection mat 200, or sensing-mat, may be placed underneath or otherwise integrated with other material layers 240a, 240b such as used in standard bed sheets. It will be appreciated that such additionally materials may confer further properties as may be required for a particular application. Typically, the conductive material of the sensors is wrapped by isolating, washable, water resistant, breathing cover mat, allowing minimum discomfort to the subject resting on the mat.

With reference now to FIGS. 2c-e showing exploded views of various embodiments of the pressure-detection mat, the conductive layers 220 (FIG. 2a) may be supported by various substrates. For example FIG. 2c shows two conductive layers 2220a, 2220b adhered directly to the insulating layer 230. Alternatively, as shown in FIG. 2d, conductive layers 3220a, 3220b may be supported by separate substrates 3210a, 3210b, such as of Thermoplastic Polyurethane (TPU) for example, the insulating layer 230 being sandwiched therebetween. In still another embodiment, as shown in FIG. 2e, the conductive layers 4220a, 4220b may themselves each be sandwiched between two substrates 4212a, 4214a, 4212b, 4214b respectively.

It will be appreciated that in order to get a stable reading of impedance values from a row of sensors, it is preferable that little or no movement be made by the subject during the taking of readings from the sensors. Accordingly, according to certain embodiments the response time of the sensors and the time taken for readings should be small possibly of the order of tens or hundreds of milliseconds, during which movement of the subject is generally insignificant although other response times may be required as appropriate. It is particularly noted that in applications where the subject is largely immobile, it may be advantageous to use longer reading times.

The pressure-detection mat, or sensing-mat is typically placed on surfaces such as a mattress of a hospital bed, a long term care facility bed, a home bed, a seat or a back of a chair, a couch, a wheelchair, or the like. Embodiments of this system can detect the pressure points formed between a subject resting on one or more pressure-detection mats and the surface upon which the mats rest. Surfaces may be parts of chairs, stools, sofas, wheelchairs, rocking chairs, chaise longue, banquets, bean bags, ottomans, benches and poufs. Pressure mapping data per subject may be aggregated over time in one or more data storage units.

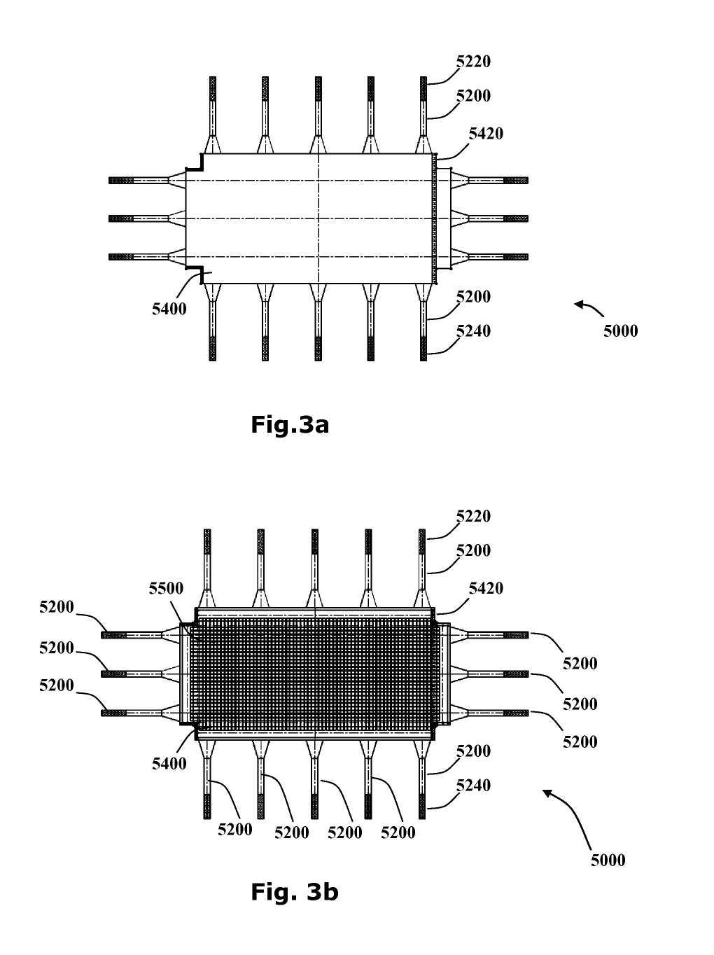

With reference to FIGS. 3a and 3b, a top view and section through respectively are shown of a further embodiment of a pressure detection mat 5000. The pressure detection mat 5000 includes a sensor matrix 5500, such as described hereinabove, housed within a cover mat 5400 and which may be sealed by a zipper 5420 as required.

The pressure detection mat 5000 may be attached to a surface in such a way that prevents movement of the mat relative to the surface. A feature of the embodiment of the mat 5000 is that the cover mat 5500 may include a coupling mechanism for securing the mat to a seat or a back of a mattress, a bed, a chair, a bench, a sofa, a wheelchair or the like. The coupling mechanism may include for example at least one strap 5200 having an attachment means 5240 configured to secure the straps 5200 to the seat or to each other such that the pressure detection mat is held securely. This may be useful to prevent folding, wrinkling or other movement of the detection mat which may contribute to the creation of shear forces which are known to encourage the formation of external pressure sores. Suitable attachment means include for example, hook-and-eye materials such as Velcro®, buckles, adhesives, buttons, laces or such like as suit requirements.

In still another embodiment, the sensor sheet may be used in a combination with an inflatable mattress optionally having a matching grid of cells. In this embodiment, when pressure exceeds a given threshold, neighboring mattress cells will inflate or deflate to redistribute the pressure. It will be appreciated that such an active solution may reduce the necessity to turn or reposition the patient. Accordingly, in certain embodiments, pressure monitoring and relief may be completely automated.

The number of pressure detection mats may vary according to need. Pressure detection mats are typically integrated to areas of a bed or a sitting apparatus which are designed to hold body parts that are prone to develop pressure-wounds. For example and without limitation, areas of a sitting apparatus may be a chair or a sofa's seats, backs, arms, back rails, restraints, leg rests or the like, which may support body parts such as but not limited to the neck, lower back, ankles or heels.

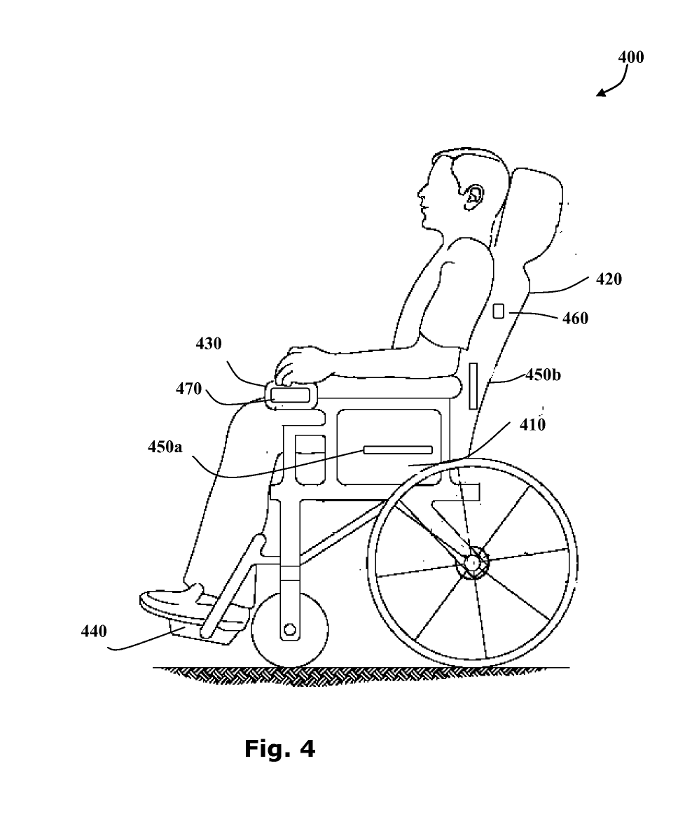

It will be appreciated that multiple embodiments of the pressure-detection mat may be located on a common sitting apparatus. Multiple embodiments of the pressure detection mat on a common sitting apparatus are demonstrated in FIG. 4, showing an embodiment of a pressure detection system integrated into a wheelchair. Embodiments of the pressure detection mats may be integrated, for example and without limitation, into the seat 410, the back 420, the arm rests 430 and the foot rests 440.

Referring back to FIG. 1a, the pressure-wound prevention system may include a power source 110 or be connected to an external power source for example and without limitation via an electric cord. In case the pressure-wound prevention system is coupled with a mobile sitting apparatus, it is important that the power source be chargeable. In electric wheelchairs, the existing battery incorporated within the electric wheelchair can further be used to supply power to the pressure-wound prevention system. In other embodiments of a sitting apparatus such as a mechanical wheelchair, a dedicated power source may be used to provide electricity to the pressure-wound prevention system. Various power sources may be usefully integrated into the system as required such as amongst others electrochemical cells, fuel cells, capacitors, solar cells, inductive power supplies, power harvesters and the like.

In various embodiments, the pressure-detection mat may further include additional sensors which can be used to detect additional environmental parameters such as temperature, humidity, ambient pressure and the like. More embodiments may further include sensors which are not integrated into the mat, aiming to detect parameters other than pressure, for example and without limitation sensors configured to detect contact between a subject and a platform. Such contact detection sensors may be placed for example and without limitation in the top rail and the cross rail of a back of a chair. Detachment of a subject from the back of the chair may result in the subject falling off the chair altogether. Therefore, information obtained from contact sensors placed in the locations mentioned earlier can be processed and used in determining whether there's danger that a subject is about to fall.

FIG. 4 illustrates how different components of a pressure-wound prevention system may be integrated into a wheelchair. The wheelchair includes a seat 410, a back 420, hand rails 430 and foot rests 440. An integrated power-source and driving unit 460 is located beneath the seat, providing power to sensing mat 450a integrated to the wheelchair seat, to a second sensing mat 450b integrated with the lower part of the back of the wheelchair, and to a touch sensor 460 located on the top rail of the wheelchair. The processing unit and the storage unit (no shown) may also located beneath the seat. A display screen 470 may be integrated into the hand rails.

In various embodiments, the data storage unit is mobile, and can be moved along with the patient from one sitting apparatus to another. Mobility of the storage unit helps preserve the pressure history of a patient as he is being moved from one room to another, or from one position to another, for example and without limitation from a hospital chair to a hospital bed or from a wheelchair to a car seat. This feature is particularly useful because moving a subject from a lying position to a sitting position does not necessarily relieve accumulated pressure applied upon all body parts.

It is a further aim of the system and method described herein to enable storage of data collected from multiple subjects in a variety of situations and a plurality of locations. Data storage is typically aggregated in one or more database units. Data storage may serve for statistics collection regarding a particular mat or line of mats, comparison of care settings according to patients' groups (for instance diabetic patients), or for the creation of a research tool designed to provide practical recommendations for turning schedules and standard of care.

### Data Analysis and Display

A software application is typically used to retrieve data from at least one data storage unit, analyze it for different purposes, and display the analysis results in various formats to a user. The software application may include features such as, but not limited to:
Calculating and presenting pressure detected by each sensor on a pressure-detection mat;
Calculating shear forces pressures by comparing relative pressures detected by adjacent pixels;
Calculating and presenting the accumulated pressure over time detected by each sensor on a pressure-detection mat;
Calculating and presenting data such as temperature or moisture build-up over time;
Calculating and alerting a caretaker at a monitoring station when patients need to be moved in order to prevent the creation of pressure-wounds;
Alarming when a pressure beyond a predefined threshold and a predefined duration is reached.
Calculating, presenting and alarming about different mat parameters, such as but not limited to wireless transmission malfunction, electricity disconnection, or the like.
Calibrating pressure-detection sensors comprising the pressure-detection mat, each sensor may be calibrated individually or a number of sensors may be calibrated in a bulk;
Configuring parameters, such as but not limited to pressure and time thresholds, for different patients or for different areas on the pressure-detection mat;
Monitoring and logging a patient's pressure-relief care routine over time;
Monitoring caretakers' performances with regard to proper treatment of patients in their care;
Translating pressure sensor readings upon the sensing mat from mat coordinates to a subject's body coordinates;
Saving historical pressure data of one or more pressure-detection mat;
Allowing visual and vocal alarms through a plurality of local and mobile devices and technologies, such as but not limited to mobile phones, beepers, personal digital assistants (PDAs), display screens in nursing stations or medical carts, web interfaces, emails, Short Messaging Service (SMS), Multimedia Messaging Service (MMS), instant text messaging platforms and the like;
Allowing a patient or his caretaker to enter data with regard to patients' care status (for instance, when the patient was last moved);
Allowing for presentation, monitoring, configuration, calculation, alarms and presentation of data from multiple pressure-detection mats used by one or more subjects; and
Enabling users to query historical pressure readings and produce reports according to their needs.

External wounds caused by tissue breakdown may develop into pressure-wounds, over time. Shear forces are a common cause of such tissue breakdown. Software may further be used to analyze data received from at least one pressure detection mat and to determine whether shear forces are exerted upon body parts of a subject. Where a subject rests upon the mat, two adjacent sensors are expected to measure approximately similar pressure levels. If that is not the case, the software may deduce that the subject is sliding upon the sensing mat and shear forces are possibly exerted upon the subject's body, creating tissue breakdown.

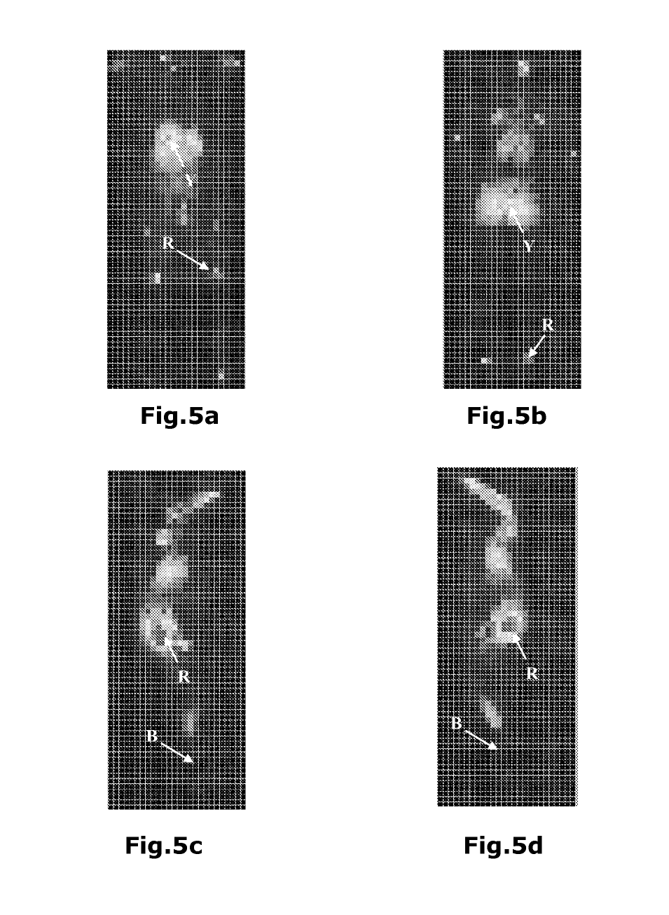

Reference is now made to FIGS. 5a-d, showing various representations of how pressure data may be displayed on a screen of an embodiment of display system 170 (FIG. 1). Respectively FIGS. 5a-d show a subject lying on his abdomen (FIG. 5a), his back (FIG. 5b), his left side (FIG. 5c) and his right side (FIG. 5d). The system shows the pressure distribution for each posture.

The display system may be a computer in communication with the data storage unit 160 (FIG. 1a), for example. Each display screen shows a matrix of pixels, each pixel representing one sensor of the pressure-detection mat. The pressure detected by each pixel is represented by a visual indication. A grayscale may be used such that higher pressures are indicated by different shades, darker grays, for example. Alternatively or additionally, colors may be used for example indicating high pressure formed between a subject's body and the surface on which the subject rests by displaying the pixel in a distinctive color, such as red (marked with R). Likewise pixels representing sensors which detect low pressure or no pressure at all may be presented in other colors such as yellow (marked with Y), blue (marked with B) or black.

Data analyzed from a pressure detection mat may be presented to at least one of a care-giver, a nurse, a man-monitored station, a friend or family member of the subject, to the subject himself or any relevant party. The display unit used to present data may be, for example and without limitation, one or more of computer screens, laptops, PDAs, cellular phone screens, printed sheets, and integrated LCD screens (e.g. TFT, touch screens).

Displaying data to more than one monitor, for example both to a family member and a hired caretaker of a subject, may assist in verification that the subject is receiving proper care from his caregiver. Displaying data to the subject himself is particularly useful in paraplegic subjects who have partial mobility. For example, a subject paralyzed from the waist down and sitting in a wheelchair may not be able to sense that a pressure-wound is forming on his abdomen. However, using the pressure-wound prevention system, he can receive a notification that accumulated pressure has been detected where his abdomen typically rests. The subject may then lean his hands on the wheelchair's arm rests and lift his abdomen off the wheelchair seat for several seconds, thus relieving pressure off the sensitive area.

Data display may include alarms. Alarms may be vocal, visual, tactile, or the like. Presentation of the alarms may be ‘local’ to the subject himself or ‘remote’ when presented to one or more users typically in charge of a subject's care, such as but not limited to a family member or a nurse at a monitoring station.

The system may further be configured to include components capable of sending data regarding the system's whereabouts, using a global positioning system (GPS) or other tracking technologies as suit requirements. For example, data such as pressure-wound formation alerts may be sent along with the system's location to a manned monitoring station. This capability may be useful, for example, when data is sent to a caretaker in charge of multiple subjects who use wheelchairs for mobility within a hospital, a nursing home or another care environment. This information can assist the caretaker in finding the subject within the care facility he resides in and provide him with proper care.

Reference is now made to FIG. 6 illustrating a flowchart of a method 600 to prevent pressure-wounds in a subject resting upon a platform. It is to be understood that unless otherwise defined, the method steps described hereinbelow can be executed either contemporaneously or sequentially in many combinations or orders of execution. Specifically, neither the ordering nor the numerals of the flowchart of FIG. 6 are to be considered as limiting. For example, two or more method steps, appearing in the following description or in the flowchart of FIG. 6 in a particular order, can be executed in a different order (e.g., a reverse order) or substantially contemporaneously.

The method commences with providing at least one pressure-detection mat comprising a plurality of pressure-detection sensors 610. The method continues with supplying electrical potential to the sensors 620, collecting data from the sensors 630, interpreting and analyzing the data collected from the sensors 640, providing an output based upon the analyzed data 650, displaying the output to at least one user 660, and optionally storing the data in at least one data storage unit 670.

It will be appreciated that the system as described hereinabove may be particularly useful in care facilities such as, amongst others, acute care facilities, sub-acute care facilities, long term care facilities, home care environments, hospices, hospitals, nursing homes, assisted living facilities and the like. In addition similar systems may be adapted for use in other environments such as hotels, vehicle seats, passenger seats, airplane seats, long-haul flight seats and the like.

The scope of the present invention is defined by the appended claims and includes both combinations and sub combinations of the various features described hereinabove as well as variations and modifications thereof, which would occur to persons skilled in the art upon reading the foregoing description.

In the claims, the word “comprise”, and variations thereof such as “comprises”, “comprising” and the like indicate that the components listed are included, but not generally to the exclusion of other components.

What is claimed is:

1. A system configured to prevent the creation of pressure-wounds in a subject, comprising:
at least one pressure detection mat comprising at least one layer of an insulating material sandwiched between a first layer of conducting strips and a second layer of conducting strips, said conducting strips of the first layer and said conducting strips of the second layer overlapping at a plurality of intersections, wherein each intersection forms a sensor;
a driving unit configured to supply electrical potential selectively to the conducting strips of the first layer;
a control unit wired to the conducting strips of the second layer and configured to control said driving unit and to receive data from said sensors;
a processor configured to monitor electrical potential on the conductive strips of the second layer, to calculate impedance values for each sensor and to determine accumulated pressure applied to said sensor by calculating a summation of pressure measured by the sensor at predetermined intervals; and
at least one display configured to present indications of pressure distribution and said accumulated pressure.

2. The system of claim 1, wherein said processor is further configured to calculate said accumulated pressure over a predetermined time period.

3. The system of claim 1, wherein said at least one display is configured to display a matrix of pixels, each said pixel representing pressure detected by one the sensors associated with the at least one pressure detection mat.

4. The system of claim 1, further comprising a platform on which the at least one pressure detection mat rests.

5. The system of claim 4, wherein the at least one pressure detection mat comprises a strap and an attachment means for securing the at least one pressure detection mat to the platform.

6. The system of claim 4, wherein said pressure detection mat is integral to said platform.

7. The system of claim 4, wherein said platform is selected from a group consisting of: mattresses, beds, chairs, stools, sofas, wheelchairs, rocking chairs, chaise lounges, bean bags, ottomans, benches and poufs.

8. The system of claim 1, wherein the at least one pressure detection mat comprises at least one substrate layer.

9. The system of claim 1, wherein at least one of said first layer of conducting strips and second layer of conducting strips are sandwiched between substrate layers.

10. The system of claim 1, wherein the sensors are selected from at least one of a group consisting of capacitance sensors, resistance sensors and impedance sensors.

11. The system of claim 1, wherein the at least one pressure detection mat further comprises at least one environmental sensor selected from a group consisting of humidity-detection sensors, temperature-detection sensors, ambient pressure sensors and combinations thereof.

12. The system of claim 1, further comprising data storage configured to store data from said processor.

13. The system of claim 12, wherein said data storage is mobile.

14. The system of claim 4, further comprising at least one contact sensor configured to detect contact between said subject and said platform.

15. The system of claim 4, wherein said processor is further configured to determine risk of said subject falling from said platform.

16. The system of claim 1, further comprising a unit configured to send data as to the whereabouts of said system to a control center.

17. The system of claim 1, wherein the processor is configured to monitor the care routine of said subject.

18. The system of claim 1, comprising a plurality of pressure detection mats in communication with at least one common control center.

19. A method for preventing the development of pressure-wounds comprising:
providing at least one pressure detection mat comprising at least one layer of an insulating material sandwiched between a first layer of conducting strips and a second layer of conducting strips, said conducting strips of the first layer and said conducting strips of the second layer overlapping at a plurality of intersections, wherein each intersection forms a sensor configured to detect accumulated pressure over time;
supplying electrical potential selectively to the conducting strips of the first layer;
monitoring electrical potential on the conductive strips of the second layer;
calculating impedance values for each sensor;
determining accumulated pressure applied to each sensor by calculating a summation of pressure measured by the sensor at predetermined intervals; and
presenting, to at least one caretaker, indications of pressure distribution and said accumulated pressure;
such that said at least one caretaker may take pressure relieving action upon said subject.

20. The method of claim 19, wherein the determining accumulate pressure further comprises performing the calculation over a predetermined time period.
